(19) United States
(12) Patent Application Publication
(10) Pub. No.: US 2021/0145918 A1
(43) Pub. Date: May 20, 2021

(54) PROBIOTIC BEVERAGES CONTAINING A CANNABINOID

(71) Applicant: HYDRO ONE LLC, Augusta, GA (US)

(72) Inventor: Babak BABAN, Augusta, GA (US)

(21) Appl. No.: 17/046,970

(22) PCT Filed: Apr. 9, 2019
(86) PCT No.: PCT/US19/26426

§ 371 Date: Oct. 12, 2020

Related U.S. Application Data
(60) Provisional application No. 62/657,037, filed on Apr. 13, 2018.
(60) Provisional application No. 62/657,038, filed on Apr. 13, 2018.

(51) Int. Cl. A61K 36/82; A61K 9/00; A61K 31/352; A61K 31/05; A61K 31/718; A61K 31/716; A61K 47/12; A61K 33/24; A61K 47/46; A61K 31/07; A61K 31/51; A61K 31/525; A61K 31/455; A61K 31/197; A61K 31/4415; A61K 31/4188; A61K 31/519; A61K 31/714; A61K 31/375; A61K 31/593; A61K 31/355; A61K 35/742; A61K 33/06; A61K 33/34; A61K 33/26; A61K 33/32; A61K 33/04; A61K 33/30; A61K 35/747; A23L 2/52; A23L 33/135; A23L 33/105; A23L 33/125; A23L 33/155; A23L 33/16; A23L 33/00; A23C 9/13

(52) U.S. Cl. CPC A61K 36/82; A23Y 2220/03; A61K 31/352; A61K 31/05; A61K 31/718; A61K 31/716; A61K 47/12; A61K 33/24; A61K 47/46; A61K 31/07; A61K 31/51; A61K 31/525; A61K 31/455; A61K 31/197; A61K 31/4415; A61K 31/4188; A61K 31/519; A61K 31/714; A61K 31/375; A61K 31/593; A61K 31/355; A61K 35/742; A61K 33/06; A61K 33/34; A61K 33/26; A61K 33/32; A61K 33/04; A61K 33/30; A61K 35/747; A23L 2/52; A23L 33/135; A23L 33/105; A23L 33/125; A23L 33/155; A23L 33/16; A23L 33/40; A23C 9/1307; A23C 9/1322; A23V 2002/00; A61K 9/0095

(57) ABSTRACT

Disclosed herein are probiotic beverages containing cannabinoid. The beverages disclosed herein may be formulated as water-based drinks or yogurt drinks, for example, and contain live, active cultures of probiotic microorganisms or may contain spores of probiotic microorganisms. The beverages may additionally include prebiotic substances to enhance the growth and colonization of the probiotic microorganisms in the intestines. The beverages have anti-inflammatory effects due to the presence of the cannabinoid but are non-narcotic. Further, the beverages serve to enhance gut health due to the presence of probiotics and optional prebiotics. The beverages are, at a minimum, composed of water, a cannabinoid, a probiotic microorganism, and optionally a tea extract, but may contain other components such as prebiotics, vitamins, minerals, other nutrients, and other natural products to provide additional health benefits and/or to improve the flavor, texture, and aroma of the beverages.

### CROSS REFERENCE TO RELATED APPLICATIONS

This application claims priority upon U.S. provisional application Ser. Nos. 62/657,037, filed Apr. 13, 2018, and 62/657,038, filed on Apr. 13, 2018. These applications are hereby incorporated by reference in their entireties for all of their teachings.

### BACKGROUND

Mammals are hosts to several hundred different species of gut microorganisms including bacteria, archaea, yeasts, and protozoa. Due to poor diet, medical treatments such as antibiotic use, and even stress or emotional factors, disruptions to the gut microbiota (known as “dysbiosis”) are increasingly common. Symptoms of dysbiosis range from gastrointestinal upset including gas, bloating, flatulence, indigestion, incomplete digestion, diarrhea, and constipation to fatigue, amenorrhea, iron deficiency, rosacea, adult acne, weakness or brittleness of the hair and nails, and more.

Meanwhile, inflammation is part of the body's immune response and can be beneficial in response to an injury. In fact, the inflammatory response is required for wound healing and repairing damage to tissue. However, chronic inflammation (i.e., in the absence of any acute stimulus including, but not limited to, a wound or cut, a crush injury, a broken bone, and the like) is increasingly a problem in modem society and has been linked to autoimmune disorders such as rheumatoid arthritis, gum disease or periodontitis, diseases of the circulatory system including atherosclerosis, allergic conditions such as hay fever, diabetes, and inflammatory bowel diseases such as ulcerative colitis and Crohn's disease. Interest in controlling inflammation is growing in both the medical community and the general population.

Many individuals find some degree of relief from dysbiosis by taking probiotic supplements containing beneficial bacteria. These supplements also occasionally contain prebiotic compounds, or indigestible fibers that provide a favorable environment for the probiotic bacteria to take root. In the case of inflammation, plant-based and natural supplements are gaining popularity as a safe and effective means to reduce inflammation and find relief from chronic conditions such as those discussed above. Further, dysbiosis and inflammation can be related conditions and are often found in the same individuals. Addressing both problems may require swallowing a large number of pills or capsules each day, which can reduce a subject's compliance with a treatment regimen.

It would be advantageous to have a single product, such as a beverage, to address both dysbiosis and inflammation. Further, it would be advantageous for the beverage to have a satisfactory taste so that the subject is encouraged to consume the beverage on a routine basis, for maximum reduction of inflammation as well as to maintain an optimal level of gut health. The present invention addresses these needs.

### SUMMARY

Disclosed herein are probiotic beverages containing cannabinoid. The beverages disclosed herein may be formulated as water-based drinks or yogurt drinks, for example, and contain live, active cultures of probiotic microorganisms or may contain spores of probiotic microorganisms. The beverages may additionally include prebiotic substances to enhance the growth and colonization of the probiotic microorganisms in the intestines. The beverages have anti-inflammatory effects due to the presence of the cannabinoid but are non-narcotic. Further, the beverages serve to enhance gut health due to the presence of probiotics and optional prebiotics. The beverages are, at a minimum, composed of water, a cannabinoid, a probiotic microorganism, and optionally a tea extract, but may contain other components such as prebiotics, vitamins, minerals, other nutrients, and other natural products to provide additional health benefits and/or to improve the flavor, texture, and aroma of the beverages.

The advantages of the invention will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION

Before the present compounds, compositions, and/or methods are disclosed and described, it is to be understood that the aspects described below are not limited to specific compounds, synthetic methods, or uses, as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an,” and “the” include plural referents unless the context clearly dictates otherwise. Thus, for example, reference to a “prebiotic” includes mixtures of two or more such prebiotics, and the like.

“Optional” or “optionally” means that the subsequently described event or circumstance can or cannot occur, and that the description includes instances where the event or circumstance occurs and instances where it does not. For example, the phrase “optionally includes a tea extract” means that the tea extract may or may not be present.

References in the specification and concluding claims to parts by weight, of a particular element or component in a composition or article, denote the weight relationship between the element or component and any other elements or components in the composition or article for which a part by weight is expressed. Thus, in a composition containing 2 parts by weight of component X and 5 parts by weight of component Y, X and Y are present at a weight ratio of 2:5, and are present in such ratio regardless of whether additional components are contained in the composition.

A weight percent of a component, unless specifically stated to the contrary, is based on the total weight of the formulation or composition in which the component is included.

As used herein, a plurality of items, structural elements, compositional elements, and/or materials may be presented in a common list for convenience. However, these lists should be construed as though each member of the list is individually identified as a separate and unique member. Thus, no individual member of any such list should be construed as a de facto equivalent of any other member of the same list based solely on its presentation in a common group, without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range was explicitly recited. As an illustration, a numerical range of “about 1 to about 5” should be interpreted to include not only the explicitly recited values of about 1 to about 5, but also to include individual values and sub-ranges within the indicated range. Thus, included in this numerical range are individual values such as 2, 3, and 4, the sub ranges such as from 1-3, from 2-4, from 3-5, etc., as well as 1, 2, 3, 4, and 5 individually. The same principle applies to ranges reciting only one numerical value as a minimum or maximum. Furthermore, such an interpretation should apply regardless of the breadth of the range or the characteristics being described.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc., of these materials are disclosed, that while specific reference to each individual and collective combination and permutation of these compounds may not be explicitly disclosed, each is specifically contemplated and described herein. For example, if a tea extract is disclosed and discussed and a number of different natural sweeteners are discussed, each and every combination of tea extract and natural sweetener that is possible is specifically contemplated unless specifically indicated to the contrary. For example, if a class of tea extracts A, B, and C are disclosed, as well as a class of natural sweeteners D, E, and F, and an example combination of A+D is disclosed, then even if each is not individually recited, each is individually and collectively contemplated. Thus, in this example, each of the combinations A+E, A+F, B+D, B+E, B+F, C+D, C+E, and C+F is specifically contemplated and should be considered from disclosure of A, B, and C; D, E, and F; and the example combination of A+D. Likewise, any subset or combination of these is also specifically contemplated and disclosed. Thus, for example, the sub-group of A+E, B+F, and C+E is specifically contemplated and should be considered from disclosure of A, B, and C; D, E, and F; and the example combination of A+D. This concept applies to all aspects of the disclosure including, but not limited to, steps in methods of making and using the disclosed compositions. Thus, if there are a variety of additional steps that can be performed with any specific embodiment or combination of embodiments of the disclosed methods, each such combination is specifically contemplated and should be considered disclosed.

The term “treat” as used herein is defined as maintaining or reducing the symptoms of a pre-existing condition (e.g., inflammation). The term “prevent” as used herein is defined as eliminating or reducing the likelihood of the occurrence of one or more symptoms of a disease or disorder.

A “pharmaceutically-acceptable compound” is used to refer to a neutral complex. In some aspects, a pharmaceutically-acceptable compound may be more economical to produce, may have increased chemical stability, may allow manipulation of the compound's pharmacokinetics and bioavailability, may make the compound easier to administer, or a combination thereof. In a further aspect, a pharmaceutically-acceptable compound can alter a compound's dissolution or solubility. In one aspect, the pharmaceutically-acceptable compound can be an ionic compound (or “pharmaceutically-acceptable salt”). For example, a pharmaceutically acceptable compound can be a reaction product between an organic acid (e.g., citric acid) and base (e.g., calcium hydroxide) to produce the ionic compound calcium citrate.

Described herein are beverages composed of a cannabinoid, a probiotic, and one or more optional components. Each component used to produce the beverages described herein and methods for producing and using the same are described below.

### Cannabinoid

The beverages described herein contain a cannabinoid. Cannabinoids are a class of molecules produced by cannabis plants, which includes over 100. The three most abundant cannabinoids include cannabidiol (CBD), tetrahydrocannabinol (THC) and cannabinodiol (CBND). In one aspect, the cannabinoid includes tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, cannabicitran, or any combination thereof. In one aspect, the cannabinoid is cannabidiol.

In one aspect, the cannabinoid is extracted from hemp seeds or is present as a hemp seed extract. In a further aspect, the cannabidiol (e.g., cannabidiol) is present in an amount of from 5 mL to 100 mL, or from 10 mL to 50 mL per 8 ounces of beverage, or the beverages contain about 5 mL, 10 mL, 15, mL, 20 mL, 25 mL, 30 mL, 35 mL, 40 mL, 45 mL, 50 mL, 60 mL, 70 mL, 80 mL, 90 mL, or 100 mL of cannabidiol per 8 ounce serving of beverage, where any value can be a lower or upper endpoint of a range (e.g., 20 mL to 40 mL, 25 mL to 35, mL, etc.).

### Probiotics

The beverages disclosed herein include a probiotic. In one aspect, the probiotic is a live microorganism that can colonize in the digestive system. In another aspect, the probiotic is a microbial spore that is shelf stable in aqueous solution and can survive the harsh environment of the upper gastrointestinal tract in order to establish a population in the gut.

In one aspect, the probiotic can be Bacillus coagulans, Lactobacillus plantarum 299v (Lp299v), or a combination thereof. In a further aspect, when the probiotic is B. coagulans, it is the strain identified as GBI-30, 6086 in the NCBI BioSample database.

In one aspect, the beverages disclosed herein include additional probiotics. In one aspect, the additional probiotic is a yeast such as, for example, an organism selected from the genera Saccharomyces, Debaromyces, Candida, Pichia, or Torulopsis. In an alternative aspect, the probiotic is a mold such as, for example, an organism selected from the genera Aspergillus, Rhizopus, Mucor, or Penicillium. In still another aspect, the probiotic is a bacterium such as, for example, an organism selected from the genera Bifidobacterium, Bacterioides, Clostridium, Fusobacterium, Melissococcus, Propionibacterium, Streptococcus, Enterococcus, Lactococcus, Kocuria, Staphylococcus, Peptostreptococcus, Bacillus, Pediococcus, Micrococcus, Euconostoc, Weissella, Aerococcus, Oenococcus, or Lactobacillus. In still another aspect, the additional probiotic is a combination of organisms from one or more of the genera listed above.

In another aspect, the additional probiotic is Aspergillus niger, A. oryzae, Bacillus coagulans, B. lentus, B. licheniformis, B. mesentericus, B. pumilus, B. subtilis, B. natto, Bacteroides amylophilus, Bac. capillosus, Bac. ruminocola, Bac. suis, Bifidobacterium adolescentis, Bif animalis, Bif breve, Bif bifidum, Bif infantis, Bif lactis, Bif longum, Bif pseudolongum, Bif. thermophilum, Candida pintolepesii, Clostridium butyricum, Enterococcus cremoris, E. diacetylactis, E. faecium, E. intermedius, E. lactis, E. mundtii, E. thermophilus, Escherichia coli, Kluyveromyces fragilis, Lactobacillus acidophilus, L. alimentarius, L. amylovorus, L. crispatus, L. bifidus, L. brevis, L. bulgaricus, L. casei, L. curvatus, L. cellobiosus, L. delbrueckii ss. bulgaricus, L. farciminis, L. fermentum, L. gasseri, L. helveticus, L. lactis, L. plantarum, L. johnsonii, L. reuteri, L. rhamnosus, L. sakei, L. salivarius, Leuconostoc mesenteroides, Pediococcus cereviseae (damnosus), P. acidlactici, P. pentosaceus, Propionibacterium freudenreichii, Prop. shertnanii, Saccharomyces cereviseae, Staphyloccus carnosus, Staph. xylosus, Streptococcus infantarius, Strep. salivarius ss. thermophilus, Strep. thermophilus, Strep. lactis, or any combination thereof.

As used herein, a “colony forming unit” or “CFU” is a measure of how many bacteria in a sample are capable of forming colonies. In this aspect, microorganisms are cultured and only viable cells are counted as CFUs.

In one aspect, the beverages disclosed herein contain from one billion to five billion CFU of the probiotic microorganism. In another aspect, the beverages contain about one, two, three, four, or five billion CFU per 8 ounces of beverage, where any value can be a lower or upper endpoint of a range (e.g., one billion to four billion, two billion to three billion, etc.)

### Teas and Tea Extracts

In certain aspects, the beverages disclosed herein can include a tea extract and/or may be a tea-based beverage. In one aspect, the tea extract is an extract of black, green, or white tea, or is a mixture of extracts of two or more types of tea. In one aspect, the beverages are caffeinated. In an alternative aspect, the beverages do not contain caffeine.

In one aspect, the extract is a white tea extract. Further in this aspect, the tea leaves from which the extract is produced exhibit little or no oxidation and retain a large proportion of their natural antioxidants. In one aspect, white tea may provide a range of health benefits including, but not limited to, improvement in oral health, anti-aging properties, antibacterial properties, reduction in the occurrence and/or severity of cardiovascular disorders, and the like. In a still further aspect, consumption of white tea may improve the symptoms of diabetes. In yet another aspect, consumption of the beverages disclosed herein by a subject confers the health benefits of white tea to the subject.

In another aspect, the extract is a green tea extract. In this aspect, the tea leaves from which the extract is produced exhibit a slightly higher degree of oxidation than white tea, but less than black tea. In one aspect, green tea may provide a range of health benefits including, but not limited to, reduction in cardiovascular risk factors, reduction in the symptoms of rheumatoid arthritis, anti-aging for the skin, immune support, increases in metabolism leading to weight loss, and/or prevention of the onset of diabetes. In a further aspect, consumption of the beverages disclosed herein by a subject confers the health benefits of green tea to the subject.

In another aspect, the extract is a black tea extract. Further in this aspect, the tea leaves from which the extract is produced have been fully oxidized. In one aspect, black tea may provide a range of health benefits including, but not limited to, improvement in oral health, reduction of blood pressure, stress reduction, reduction in the occurrence and/or severity of digestive problems, and the like. In another aspect, consumption of particular flavonoids in black tea can reduce fracture risk in osteoporotic women. In yet another aspect, consumption of the beverages disclosed herein confers the health benefits of black tea to the subject.

In one aspect, the beverages disclosed herein include from 0.1% to 1% by volume of the beverage of tea extract per 8 ounces of beverage. In a further aspect, the beverages contain from 0.1 to 1%, or from 0.25 to 0.75%, or from 0.3 to 0.5%, or about 0.4% by volume tea extract per 8 ounces of beverage. In one aspect, the beverages contains about 0.4% by volume decaffeinated tea extract per 8 ounces of beverage.

### Prebiotics

In another aspect, the beverage further contains a prebiotic. As used herein, “prebiotic” refers to a food ingredient that promotes the growth of beneficial microorganisms (such as, for example, microorganisms consumed as probiotics) in the intestine. In one aspect, the prebiotic can be an inulin or can be maltodextrin, or a combination thereof. In one aspect, the maltodextrin, when included, is a non-GMO digestion-resistant maltodextrin.

In one aspect, the prebiotic is derived from acacia gum or gum arabic, raw dandelion greens, raw leeks, Jerusalem artichokes, raw jicama, underripe bananas, raw chicory root, raw garlic, onions, agave nectar, raw wheat bran, whole wheat flour, yacon, Echinacea, burdock, globe artichoke, wild yam, mugwort, or raw asparagus. In one aspect, the prebiotic is or contains an inulin. In another aspect, the prebiotic can be a “galactooligosaccharide” or “GOS.”

As used herein, “inulin” refers to a naturally occurring polysaccharide produced by a plant such as, for example, chicory. An inulin is a type of dietary fiber and is classified as a “fructan” or a polymer of fructose. A “fructooligosaccharide” or “FOS” or “oligofructose” is a fructan with a short chain length. As an example, the degree of polymerization in inulin can be from 10 to 60, or in some instances, can be up to several thousand fructose units, whereas the degree of polymerization in FOS produced from the chemical degradation of inulin can be from 1 to 14 or from 1 to 7. FOS may improve the flavor or odor of food products; FOS is often used as alternative sweeteners, but FOS, like inulin, can serve as a substrate for probiotic microorganisms in the large intestine.

As used herein, “maltodextrin” is a polysaccharide produced from plant starches such as, for example, corn or wheat starches. In a further aspect, in addition to acting as a prebiotic, when included in the beverages disclosed herein, maltodextrin can alter the thickness or mouthfeel of the beverages. In a still further aspect, when included in the beverages disclosed herein, the maltodextrin can add sweetness to the beverages, or may remain largely flavorless.

In one aspect, the beverages described herein are “symbiotic.” This term refers to products containing both probiotics and prebiotics and specifically to products wherein the prebiotics are chosen selectively to favor the growth of the probiotic microorganisms.

### Organic Acid

In some aspects, the beverages disclosed herein include an organic acid. In one aspect, the organic acid is the free acid. In an alternative aspect, the organic acid is a pharmaceutically-acceptable salt. In a further aspect, the organic acid is malic acid, citric acid, a combination thereof, or the pharmaceutically-acceptable salt of any of these.

In another aspect, the organic acid is present in an amount of from 0.01% to 0.1% by volume of the beverages disclosed herein. In a further aspect, the beverages contain 0.01, 0.02, 0.03, 0.04, 0.05, 0.06, 0.07, 0.08, 0.09, or 0.1% by volume of the organic acid per 8 ounces of beverage. In another aspect, the beverages contain from 0.01 to 0.06% by volume of the organic acid, or from 0.02 to 0.05% by volume of the organic acid per 8 ounces of beverage.

### Vitamins

As used herein, a vitamin is an organic compound that is required in small quantities to support essential body functions including growth and cellular maintenance. In some aspects, vitamins cannot be produced by the body (e.g., vitamin C) or are not produced in sufficient quantities to meet a patient's needs and must be supplied via external sources. In one aspect, many diets are insufficient in one or more key vitamins and supplements are often indicated. In one aspect, a vitamin can act as a cofactor to assist an enzyme in carrying out an essential biochemical reaction. In one aspect, the beverages disclosed herein include one or more vitamins. In a further aspect, the vitamin can be vitamin A, B₁, B₂, B₃, B₅, B₆, B₇, B₉, B₁₂, C, D₃, E, or K. In a still further aspect, an 8 ounce serving of the beverage contains 100% of the recommended daily intake of any vitamins contained therein.

### Vitamin A

In one aspect, the beverages disclosed herein include vitamin A. Vitamin A can be important for maintenance of the immune system and for low-light and color vision. Vitamin A can also act as an antioxidant. In one aspect, the vitamin A source is retinyl palmitate, which is converted to retinol by the body, or is beta-carotene, or a combination thereof. In one aspect, the amount of retinyl palmitate or other retinol source per 8 ounce serving of the beverages provides the from 500 to 1500 μg of vitamin A, or from 750 to 1000 μg of vitamin A, or from 700 to 900 μg of vitamin A. In another aspect, the amount of beta carotene per 8 ounce serving of the beverages can be higher than 1500 μg because the subject's body converts only the amount needed to retinol. In still another aspect, a mixture of the retinol and beta-carotene forms of vitamin A is included in the beverages disclosed herein.

### B Complex Vitamins

In one aspect, the beverages disclosed herein include one or more B vitamins. B vitamins generally act as cofactors or coenzymes or precursors needed to make cofactors or coenzymes. B vitamins are not stored in the body and must be regularly supplied by dietary or other means to avoid deficiency.

Low levels of B vitamins can affect the manufacture of neurotransmitters and contribute to stress and anxiety. Here, supplementation of B vitamins can reduce stress related to their deficiency. Low levels of B vitamins can be caused by kidney dialysis, the use of diuretics, and overconsumption of refined carbohydrates, since digestion of many carbohydrates requires the use of B vitamins. Inadequate B vitamin intake is linked to blood sugar surges in patients who consume too many refined carbohydrates.

In one aspect, thiamin or vitamin B₁ is included in the beverages disclosed herein. Thiamin may be important to nerve and muscle health, production of hydrochloric acid in the stomach, and treatment of constipation and fatigue, as well as assisting digestion of some carbohydrates and proteins. In one aspect, the thiamine source is thiamine mononitrate. In one aspect, the amount of vitamin B₁ present per 8 ounce serving of the beverages is from 0.1 mg to 50 mg, 0.1 mg to 25 mg, 0.1 mg to 10 mg, 0.1 mg to 5 mg, 0.1 mg to 2 mg, or 0.1 mg to 1 mg. In another aspect, 0.5 mg to 2 mg of thiamin is present per 8 ounce serving of the beverages.

In another aspect, riboflavin, or vitamin B₂, is included in the beverages disclosed herein. Riboflavin may be important for growth, red blood cell production, and eye health, as well as assisting in the digestion of some carbohydrates, fats, ketone bodies, and proteins. In one aspect, the riboflavin source is molecular riboflavin. In one aspect, the amount of vitamin B₂ present per 8 ounce serving is from 0.1 mg to 2 mg. 0.5 mg to 2 mg, or 0.5 mg to 1 mg. In another aspect, 0.5 to 2 mg or riboflavin is present per 8 ounce serving of the beverages.

In still another aspect, nicotinic acid or nicotinamide, also known as niacinamide or vitamin B₃, is included in the beverages disclosed herein. Vitamin B₃ is important to digestive system health and may assist in the digestion of some carbohydrates, as well as in the production of various sex and stress-related hormones. Niacin may be useful in reducing cholesterol levels in the blood. In one aspect, the niacinamide source is molecular niacinamide. In a further aspect, the amount of vitamin B₃ present per 8 ounce serving is from 1 mg to 40 mg, 5 mg to 30 mg, or 5 mg to 25 mg. In another aspect, 5 to 25 mg of niacinamide is present per 8 ounce serving of the beverage.

In one aspect, pantothenic acid, also known as vitamin B₅, is included in the beverages disclosed herein. Vitamin B₅ is important in the metabolism of macronutrients such as proteins, fats, and carbohydrates, and is required for the synthesis of coenzyme A. Pantothenic acid is also important for acylation and acetylation in signal transduction and other biological functions including enzyme activation. In one aspect, the pantothenic acid source is pantothenol or calcium pantothenate. In a further aspect, the amount of vitamin B₅ present per 8 ounce serving is from 1 mg to 10 mg, or from 2 mg to 8 mg, or from about 4 to 5 mg. In one aspect, 5 mg of pantothenic acid is present per 8 ounce serving of the beverage.

In still another aspect, the beverages disclosed herein include pyridoxal phosphate (also occurring as pyridoxine), or vitamin B₆. Pyridoxal phosphate may be important for brain health and the production of red blood cells and immune system cells. In a further aspect, deficiencies in vitamin B₆ have been linked to diabetes, nervous system disorders, and heart disease. In one aspect, the pyridoxine source is pyridoxine hydrochloride. In one aspect, the amount of vitamin B₆ present per 8 ounce serving is from 1 mg to 100 mg, 1 mg to 50 mg, or 1 mg to 10 mg. In another aspect, 1 to 10 mg of pyridoxine hydrochloride is present per 8 ounce serving of the beverage.

In still another aspect, the beverages disclosed herein include biotin, or vitamin B₇. Biotin is important to various aspects of metabolism and may be important in strengthening the hair and nails as well as in the metabolism of fats and amino acids. In one aspect, the biotin source is molecular biotin. In one aspect, the amount of vitamin B₇ present per 8 ounce serving is from 10 μg to 500 μg, 10 μg to 300 μg, or 10 μg to 100 μg. In another aspect, 10 μg to 100 μg of biotin is present per 8 ounce serving of the beverage.

In one aspect, the beverages disclosed herein include folate, also known as folic acid or vitamin B₉. Folic acid may be important for brain function, mental health, red blood cell production, and production of nucleic acids. In one aspect, the folate source is folic acid. In a further aspect, the amount of vitamin B₉ present per 8 ounce serving of the supplement is from 0.05 mg to 1 mg, 0.1 mg to 1 mg, or 0.1 mg to 0.5 mg. In another aspect, 0.2 to 0.4 mg of folic acid is present per 8 ounce serving of the beverage.

In another aspect, cobalamin, or vitamin B₁₂, is included in the beverages disclosed herein. Cobalamin may be present as the cyanocobalamin, hydroxycobalamin, adenosylcobalamin, or methylcobalamin form. Cobalamin is important to nervous system health, the production of red blood cells, and synthesis of nucleic acids. In a further aspect, cobalamin works synergistically with folate with respect to red blood cell production. In one aspect, the cobalamin source is cyanocobalamin. In a further aspect, the amount of vitamin B₁₂ present per 8 ounce serving of the beverage is from 1 μg to 150 μg, 1 μg to 100 μg, 1 μg to 50 μg, 1 μg to 25 μg, or 1 μg to 10 μg. In another aspect, 3 to 6 μg of cyanocobalamin is present per 8 ounce serving of the beverage.

### Vitamin C

In one aspect, the beverages disclosed herein include ascorbic acid or vitamin C. Vitamin C is important to the immune system, collagen production, and wound healing. Further in this aspect, vitamin C is needed for the biosynthesis of hydroxyproline, which is important in the synthesis of collagen, osteoid, and dentin. Vitamin C is a potent antioxidant that can fight free radical damage. Finally, vitamin C may assist with the uptake of non-heme iron. In yet another aspect, vitamin C helps to protect folate from oxidative damage.

In one aspect, the amount of vitamin C present per 8 ounce serving of the beverage is from 10 mg to 2,000 mg, 10 mg to 1,500 mg, 10 mg to 1,000 mg, 100 to 500 mg, or 100 mg to 300 mg. In another aspect, 100 to 300 mg of vitamin C (as ascorbic acid) is provided per 8 ounce serving of the beverages.

### Vitamin D

In one aspect, the beverages disclosed herein include vitamin D. Vitamin D is a fat-soluble vitamin that is usually activated by healthy or well-functioning kidneys. Vitamin D may improve bone health and immune system function and may protect against certain cancers. Vitamin D can increase calcium and phosphate absorption from the small intestine, is important in bone mineralization, and maintains proper calcium and phosphorus levels in the serum. In one aspect, the beverages disclosed herein include a source of vitamin D. In a further aspect, the source of vitamin D is vitamin D₃, or cholecalciferol.

In one aspect, the amount of vitamin D present per 8 ounce serving of the beverage is from 1 μg to 100 μg, 1 μg to 50 μg, or 1μg to 20 μg. In another aspect, 5 to 10 μg of vitamin D (as cholecalciferol) are provided per 8 ounce serving of the beverages disclosed herein.

### Vitamin E

In one aspect, the beverages disclosed herein include vitamin E. Vitamin E is a fat-soluble vitamin found naturally in plant oils such as corn oil, soybean oil, wheat germ oil, sunflower oil, and safflower oil. Vitamin E can act as an antioxidant and can neutralize reactive oxygen species that would otherwise cause cell or tissue damage. Vitamin E deficiency, although rare, can cause various disorders of neural and nervous tissues including macular degeneration, retinopathy, and poor conduction of electrical impulses along nerves. Vitamin E may also protect lipids, including cell membrane lipids, from oxidation. Finally, vitamin E may have a role in reducing inflammation. In one aspect, the vitamin E included herein can be in the form of a tocopherol (α, β, γ, or δ), a tocotrienol (α, β, γ, or δ), or a combination of any of these. In one aspect, the vitamin E is extracted from a plant source.

In one aspect, the amount of vitamin E present per 8 ounce serving of the beverage is from 5 to 30 mg, or is from 10 to 20 mg, or is about 15 mg. In a further aspect, 15 mg of vitamin E (as mixed tocopherols) are provided per 8 ounce serving of the beverages disclosed herein.

### Vitamin K

In one aspect, the beverages disclosed herein include vitamin K. Vitamin K is a fat-soluble vitamin required for synthesis of proteins involved in blood coagulation and/or for controlling calcium binding in tissues. In one aspect, the beverages include vitamin K₁ or phylloquinone, which is converted to vitamin K₂ by gut bacteria. In an alternative aspect, the beverages include vitamin K₂ or menaquinone. In some aspects, vitamin K supplementation is contraindicated (such as for patients taking warfarin) and so, in some aspects, the beverages do not include a source of vitamin K.

In one aspect, when included, the amount of vitamin K present per 8 ounce serving of the beverage is from 50 to 150 μg of vitamin K, or is about 75 to 125 μg of vitamin K, or is from about 100 to 120 μg of vitamin K.

### Minerals

As used herein, a mineral is an inorganic element that is obtained from food or supplementation and is required for the functioning of the human body. Minerals include, but are not limited to, calcium, chromium, copper, iron, magnesium, manganese, molybdenum, nickel, potassium, selenium, sodium, vanadium, cobalt, and zinc. In one aspect, a mineral can act as a cofactor. In other aspects, minerals can be used for cell signaling, or can be essential structural components of the body (e.g., calcium in bone), or can associate with proteins, nucleic acids, lipids, and carbohydrates to maintain particular secondary, tertiary, and quaternary structures.

In another aspect, the mineral can be part of a chelate complex. Chelates have a cyclic structure in which a central metallic ion is held tight via covalent-coordinate bonds to form a coordinate compound or chelate complex. Furthermore, chelates occur abundantly in nature; for example, chlorophyll is a chelate of magnesium, hemoglobin of iron, vitamin B₁₂ with cobalt, hemocyanin with copper, and enzymes that contain vanadium or molybdenum.

### Calcium

In one aspect, the beverages disclosed herein include a pharmaceutically-acceptable compound of calcium. Calcium supplementation can be useful in preventing osteoporosis since calcium is a major component of bones and teeth. Calcium's absorption and effects are enhanced or aided by vitamin D and parathyroid hormone. Calcium may be important to B complex vitamin absorption and is important to the functional integrity of many cells. Moreover, calcium requirements may increase with increased protein consumption.

In one aspect, the calcium is provided as an ionic compound of calcium. Examples of such compounds include, but are not limited to, calcium citrate, calcium citrate tetrahydrate, calcium lactate pentahydrate, and calcium ascorbate. In one aspect, the beverages disclosed herein include an ionic compound of calcium in an amount such that there are 100 to 2,500 mg, 100 to 2,000 mg, 100 to 1,500 mg, 100 to 1,000 mg, or 100 to 750 mg of calcium per 8 ounce serving of the beverages. For example, calcium citrate tetrahydrate has a molecular weight of 570.49, of which calcium is 21.08%. Thus, in order for the beverage to have 500 mg of calcium per serving, there will be approximately 2,372 mg of calcium citrate tetrahydrate per serving. In another aspect, 250 to 500 mg of calcium is provided per 8 ounce serving of the beverages disclosed herein.

### Chromium

In one aspect, the beverages disclosed herein include a pharmaceutically-acceptable compound of chromium. Chromium is required in trace amounts in the human diet. In a still further aspect, trivalent (3+) chromium is the biologically active and non-toxic oxidation state of chromium. Chromium can enhance the action of insulin and may be involved in the metabolism of macronutrients including carbohydrates, protein, and fats.

In one aspect, the chromium is provided as an ionic compound of chromium or as an extract from a natural source of chromium. Further in this aspect, the chromium can be provided as chromium chloride, chromium nicotinate, chromium picolinate, high-chromium yeast, or chromium citrate. In one aspect, the chromium is in the form of chromium picolinate. In one aspect, the beverages disclosed herein include from 1 to 10 μg of chromium, or from 1 to 6 μg of chromium, or from 1.25 to 5.25 μg of chromium, or from 1.75 to 3.75 μg of chromium per 8 ounce serving of beverage.

### Cobalt

In another aspect, the beverages disclosed herein include a pharmaceutically-acceptable compound of cobalt. Cobalt is a central component of cobalamin or vitamin B₁₂. The primary biological function of cobalt relates to the function of vitamin B₁₂ as discussed above.

In one aspect, the cobalt is provided as part of a cobalamin compound, wherein the cobalt is complexed by four nitrogen atoms and an additional substituent which may be a cyano group (as in cyanocobalamin), a hydroxyl group (as in hydroxocobalamin), a methyl group (as in methylcobalamin), or an adenosyl group (as in adenosylcobalamin). In one aspect, the cobalt source is adenosylcobalamin or methylcobalamin. In a further aspect, the amount of cobalt included in the beverages disclosed herein is dictated by the amount of vitamin B₁₂ included as outlined previously.

### Copper

In one aspect, the formulations disclosed herein include a pharmaceutically-acceptable compound of copper. Biological copper is important in electron transport and oxygen transport and is found in cytochrome c oxidase in mitochondria. Copper is a component of superoxide dismutases and may aid in iron absorption.

In one aspect, the copper is provided as an ionic compound of copper. Further in this aspect, the copper can be provided as copper oxide, copper chloride, copper gluconate, copper sulfate, or a copper amino acid chelate. In one aspect, the chromium is in the form of cupric oxide. In one aspect, the beverages disclosed herein include from 100 to 200 μg of copper, or from 500 to 1500 μg of copper, or from 800 to 1000 μg of copper, or about 900 μg of copper per 8 ounce serving of beverage.

### Iron

In one aspect, the formulations disclosed herein include a pharmaceutically-acceptable compound of iron. Iron is vital in preventing anemia. Iron is biochemically important to oxygen and electron transport and may improve the quality and quantity of red blood cells. Heme iron is found in meats and is well-absorbed, while non-heme iron is poorly absorbed. Supplementation of iron may be particularly important for patients consuming diets that include little or no meat.

In one aspect, the iron is provided as an ionic compound of iron such as, for example, iron gluconate, iron gluconate dehydrate, or iron sulfate heptahydrate. In one aspect, the beverages disclosed herein include an ionic compound of iron in an amount such that there are 1 mg to 45 mg, 1 mg to 30 mg, 1 mg to 20 mg, or 5 mg to 20 mg of iron per 8 ounce serving.

### Magnesium

In one aspect, the beverages disclosed herein include a pharmaceutically-acceptable compound of magnesium. Magnesium is necessary for the proper functioning of calcium in the body such as, for example, assisting in entry of calcium ions into cells, thus preventing calcification of tissues. Magnesium supplementation may support healthy heart function.

Magnesium is required for normal bone structure formation and the functioning of several hundred enzymes, especially those with ATP-dependent phosphorylation, protein synthesis, and carbohydrate metabolism functions. Magnesium in the extracellular matrix is important to electrical potentials in nerve and muscle cells and the transmission of impulses across neuromuscular junctions.

In one aspect, the magnesium is provided as an ionic compound of magnesium. Examples of such compounds include, but are not limited to, magnesium citrate, magnesium sulfate monohydrate or heptahydrate, magnesium acetate tetrahydrate, magnesium D-gluconate hydrate, or magnesium nitrate hexahydrate. In one aspect, the beverages described herein include an ionic compound of magnesium in an amount such that there are 100 to 500 mg, 150 to 500 mg, 200 to 500 mg, 250 to 500 mg, or 300 to 500 mg of magnesium per 8 ounce serving of the beverages.

### Manganese

In one aspect, the beverages disclosed herein include a pharmaceutically-acceptable compound of manganese. Manganese is a required cofactor for various oxidoreductases, transferases, hydrolases, lyases, isomerases, ligases, lectins, and integrins, as well as a magnesium-containing superoxide dismutase. Manganese is also an important mineral for bone health including in cartilage formation and bone mineralization.

In another aspect, the manganese is provided as an ionic compound of manganese. Examples of such compounds include, but are not limited to, manganese amino acid chelates, manganese ascorbate, manganese chloride, manganese chloride tetrahydrate, manganese citrate, manganese dioxide, manganese gluconate, manganese glycerophosphate, manganese sulfate, manganese sulfate monohydrate, and manganese sulfate tetrahydrate. In one aspect, the beverages disclosed herein include an ionic compound of manganese such that there are 1 to 5 mg, or 1 to 4 mg, or 1.5 to 2.5 mg, or 1.8 to 2.3 mg, or about 2 mg of manganese per 8 ounce serving of beverage.

### Molybdenum

In one aspect, the beverages disclosed herein include a pharmaceutically-acceptable salt of molybdenum. Molybdenum is a heteroatom at the active site of certain enzymes such as, for example, sulfite oxidase, xanthine oxidoreductase, aldehyde oxidase, and mitochondrial amidoxime reductase, and molybdenum is used by the body in catabolism of proteins. Lack of molybdenum in the diet has been associated with higher rates of esophageal cancer.

In one aspect, the molybdenum is provided as an ionic compound of molybdenum or molybdate including, but not limited to, ammonium molybdate, sodium molybdate, molybdenum citrate, or molybdenum picolinate. In one aspect, the beverages disclosed herein include an ionic compound of molybdenum or molybdate such that there are 10 to 100 μg, or 25 to 75 μg, or 30 to 50 μg, or about 45 μg of molybdenum per 8 ounce serving of the beverage.

### Nickel

In one aspect, the beverages disclosed herein include a pharmaceutically-acceptable salt of nickel. Nickel acts as an essential nutrient for bacteria residing in the large intestine, and is an important cofactor for several bacterial enzymes. Nickel may be important for increasing iron absorption and preventing anemia, and may also have a role in strengthening osteoporotic bones.

In one aspect, the nickel is provided as an ionic compound of nickel including, but not limited to, nickel chloride or nickel sulfate. In a further aspect, the beverages disclosed herein include an ionic compound of nickel such that there are from 0.1 to 1 mg of nickel, or from 0.25 to 0.75 mg of nickel, or from 0.3 to 0.6 mg of nickel, or about 0.6 mg of nickel per 8 ounce serving of the beverage.

### Potassium

In one aspect, the formulations disclosed herein include a pharmaceutically-acceptable salt of potassium. A diet low in potassium can put one at risk for hypertension and/or hypokalemia. In a still further aspect, potassium supplementation can decrease blood pressure in people with hypertension and can reduce the risk of stroke.

In another aspect, the potassium is provided as an ionic compound of potassium including, but not limited to, potassium acetate, potassium bicarbonate, potassium chloride, potassium citrate, potassium gluconate, potassium glycerophosphate, potassium orotate, potassium phosphate, potassium ascorbate, potassium hydroxide, or potassium sulfate. In a still further aspect, the beverages disclosed herein include an ionic compound of potassium such that there are from 2.5 to 7.5 g of potassium, or from 4 to 6 g of potassium or from 4.7 to 5.4 g of potassium, or about 4.7 g of potassium per 8 ounce serving of the beverage.

### Selenium

In one aspect, the beverages disclosed herein include a pharmaceutically-acceptable compound of selenium. Selenium is a powerful antioxidant and is especially useful when employed against the damaging effects of free radicals. Selenium supplementation may protect against hardening of the arteries and harmful molecules.

In one aspect, the selenium is provided as an ionic compound of selenium such as, for example, sodium selenate. In a further aspect, the formulations described herein include an ionic compound of selenium in an amount such that there are 10 μg to 400 μg, 10 μg to 300 μg, 10 μg to 200 μg, 10 μg to 100 μg, or 25 μg to 80 μg of selenium per 8 ounce serving of beverage.

### Sodium

Sodium is an essential mineral that regulates blood pressure and blood volume. However, sodium is a major contributor to hypertension and is found in excess in many Western diets. In one aspect, the beverages disclosed herein do not include sodium. In another aspect, the beverages disclosed herein include no more than 500 mg of sodium, or include less than 50 mg of sodium, or include essentially 0 mg of sodium per 8 ounce serving of the beverages.

### Vanadium

In one aspect, the beverages disclosed herein include a pharmaceutically-acceptable compound of vanadium. Vanadium can be used for treating diabetes and prediabetes, edema or water retention, heart disease, and high cholesterol. Vanadium can act like insulin or helps enhance the effects of insulin.

In another aspect, the beverages disclosed herein are supplemented with a pharmaceutically-acceptable salt of vanadium such as, for example, vanadium pentoxide, vanadyl sulfate, or vanadyl nicotinate. In a further aspect, the beverages disclosed herein include an ionic compound of vanadium containing from 0.25 to 1.5 mg of vanadium, or from 0.25 to 1 mg of vanadium, or from 0.25 to 0.5 mg of vanadium.

### Zinc

In one aspect, the beverages disclosed herein include a pharmaceutically-acceptable compound of zinc. Zinc can have anti-inflammatory and anti-depressant effects or may support the immune system. Zinc is integral to the function of many metalloenzymes and is a cofactor in the synthesis of nucleic acids. Zinc is also important in the mobilization of vitamin A from the liver and in several reproductive system hormones in both men and women.

In one aspect, the zinc is provided as an ionic compound of zinc. Examples of such compounds include, but are not limited to, zinc citrate, zinc citrate dehydrate, zinc acetate dehydrate, or zinc nitrate hexahydrate. In one aspect, the beverages disclosed herein include an ionic compound of zinc in an amount such that there is 1 mg to 40 mg, 1 mg to 20 mg, or 5 mg to 20 mg of zinc per 8 ounce serving of beverage.

### Additional Components

In some aspects, the beverages disclosed herein include additional ingredients known in the art. In other aspects, the beverages are 100% natural (i.e., made with 100% natural components) with no artificial colors, dyes, preservatives, sweeteners, or the like.

In one aspect, the beverages are formulated with no ingredients from genetically-modified organisms. In another aspect, the beverages are gluten free, lactose/dairy free, and vegan.

In one aspect, the additional ingredients are selected from polydextrose, milk protein concentrate, sodium citrate, malic acid, tapioca starch, corn starch, calcium caseinate, potassium carbonate, vegetable juice, salt or sea salt, carrageenan, whey protein concentrate, fruit juice concentrate, cane sugar or another sweetener, chicory root fiber, fruit pectin, locust bean gum, milk, natural flavors, preservatives, or any combination thereof. In another aspect, the additional ingredients are selected from one or more of the following categories:

### Sweetener

A “sweetener” is a compound, extract, or composition that imparts a sweet taste to a food of beverage. Inclusion of a sweetener may assist in improving the flavor of a food or beverage product. In one aspect, the beverages disclosed herein do not contain any added sweeteners. In another aspect, the beverages disclosed herein contain one or more added sweeteners.

In one aspect, the sweetener is a natural sweetener. Examples of sweeteners useful herein include, but are not limited to, cane sugar, beet sugar, coconut sugar, agave nectar, honey, maple syrup, molasses, barley malt syrup, brown rice syrup, date sugar, xylitol, erythritol, lucuma powder, monk fruit extract, stevia extract, or any combination thereof. In one aspect, the sweetener can add calories or other nutrients such as, for example, fiber to the beverages. In an alternative aspect, the sweetener is a non-caloric sweetener.

In one aspect, the sweetener or other additional ingredients adds a small number of calories to the beverages disclosed herein. In one aspect, the beverage has from 5 calories to 20 calories per 8 ounce serving, or from 10 calories to 20 calories per serving, or from 10 calories to 15 calories per serving, or has about 10 calories per serving.

### Preservative

A “preservative” is a compound or composition that is added to food and/or beverage products to prevent spoilage and prolong shelf life. In one aspect, a preservative can be an artificial or added preservative, such as, for example, sodium benzoate, benzoic acid, a nitrite, a sulfite, sodium sorbate, potassium sorbate, or a similar compound. In one aspect, added preservatives are not found in natural products. In another aspect, the beverages described herein do not contain any added or artificial preservatives.

In one aspect, the beverages described herein include a natural preservative. “Natural preservatives” as described herein include antioxidants such as, for example, vitamins A, C, and E (often present as “mixed tocopherols”). In another aspect, the natural preservative can be rosemary oil or rosemary leaf extract, lemon juice, cinnamon extract, citric acid, or a combination thereof.

### Natural Flavors

“Natural flavors” are defined by the Code of Federal Regulations as essential oils, oleoresins, essences or extractives, protein hydrolysates, distillates, or any products of roasting, heating, or enzymolysis, which contain the flavoring constituents derived from a spice, fruit or fruit juice, vegetable or vegetable juice, edible yeast, herb, bark, bud, root, leaf, or similar plant material, or meat, seafood, poultry, eggs, dairy products, or fermentation products thereof, whose significant function in food is flavoring rather than nutritional. In one aspect, the beverages disclosed herein include one or more added natural flavors. In an alternative aspect, the beverages disclosed herein are free from any added natural flavors other than those already possessed by the ingredients described herein.

### Properties and Manufacture of the Beverages

In one aspect, the beverages disclosed herein are free of THC and gluten. In another aspect, the beverages are kosher and/or halal. In still another aspect, the beverages are free of genetically-modified organisms (GMOs), are lactose free, and/or are vegan.

In one aspect, the components of the beverages disclosed herein can be admixed in any order using techniques known in the art. “Admixing” or “admixture” refers to a combination of two components together when there is no chemical reaction or physical interaction. The terms “admixing” and “admixture” can also include the chemical interaction or physical interaction between any of the components described herein upon mixing to produce the composition. The components can be admixed alone or in water.

In one aspect, the beverages disclosed herein require refrigeration before and/or after opening. In another aspect, the beverages disclosed herein do not require refrigeration.

In one aspect, the beverages are composed primarily of water, which is referred to herein as “water-based beverage.” In one aspect, the beverage is at least 90% by volume, at least 95% by volume, or at least 98% by volume of the beverage.

In one aspect, the water-based beverage comprises water, cannabidiol in the amount of from 10 mL to 50 mL per 8 ounce serving, and a probiotic comprising Bacillus coagulans in the amount of one billion CFU to five billion CFU per 8 ounce serving.

In a further aspect, the water-based beverages optionally comprise a tea extract in the amount of from 0.1% to 1% by volume per 8 ounce serving. In a still further aspect, the beverages optionally comprise a prebiotic such as, for example, an inulin or maltodextrin.

In yet another aspect, the water-based beverages further contain an organic acid such as citric acid or malic acid or a combination thereof; a vitamin or vitamins such as, for example, vitamin A (as retinyl palmitate), B₁, B₂, B₃ (as niacinamide), B₅ (as D-calcium pantothenate), B₆ (as pyridoxine hydrochloride), B₇, B₉, B₁₂ (as cyanocobalamin), C, D₃, E (as D-alpha tocopheryl acetate), or any combination thereof; and/or a mineral such as, for example, calcium, chromium (as chromium picolinate), copper, iron, magnesium, manganese, molybdenum, nickel, potassium, selenium, vanadium, cobalt (as cyanocobalamin), zinc, or a combination thereof.

In a first aspect, the water-based beverage comprises (a) water, (b) a cannabinoid, (c) a probiotic, and (d) optionally a tea extract.

In a second aspect, the beverage of the first aspect includes the tea extract.

In a third aspect, wherein the tea extract of the second aspect is from 0.1% to 1% by volume of the beverage per 8 ounces of beverage.

In a fourth aspect, the cannabinoid of the first aspect comprises tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, cannabicitran, or any combination thereof.

In a fifth aspect, the cannabinoid of the first aspect is cannabidiol.

In a sixth aspect, the cannabinoid of the fifth aspect is from 10 mL to 50 mL per 8 ounces of beverage.

In a seventh aspect, the cannabinoid of the sixth aspect is cannabidiol.

In an eighth aspect, the probiotic of the first aspect comprises Bacillus coagulans, Lactobacillus plantarum 299v (Lp299v), or a combination thereof.

In a ninth aspect, the probiotic of the first aspect is from one billion CFU to five billion CFU per 8 ounces of beverage.

In a tenth aspect, the beverage of the first aspect further comprises a prebiotic.

In an eleventh aspect, the first aspect the prebiotic of the tenth aspect comprises an inulin or maltodextrin.

In a twelfth aspect, the beverage of the first aspect further comprises an organic acid or a pharmaceutically-acceptable salt thereof.

In a thirteenth aspect, the organic acid of the twelfth aspect is from 0.01% to 0.1% by volume of the beverage per 8 ounces of beverage the first aspect.

In a fourteenth aspect, the organic acid of the twelfth aspect comprises malic acid or a pharmaceutically-acceptable salt thereof, citric acid or a pharmaceutically-acceptable salt thereof, or a combination thereof the first aspect.

In a fifteenth aspect, the beverage of the first aspect further comprises one or more vitamins.

In a sixteenth aspect, the vitamin of the fifteenth aspect comprises vitamin A, B₁, B₂, B₃, B₅, B₆, B₇, B₉, B₁₂, C, D₃, E, or any combination thereof the first aspect

In a seventeenth aspect, wherein the beverage of the first aspect further comprises one or more minerals.

In an eighteenth aspect, the mineral of the seventeenth aspect comprises a pharmaceutically-acceptable salt of calcium, chromium, copper, iron, magnesium, manganese, molybdenum, nickel, potassium, selenium, sodium, vanadium, cobalt, zinc, or any combination thereof.

In a nineteenth aspect, the mineral of the seventeenth aspect comprises chromium picolinate.

In a twentieth aspect, the beverage of the first aspect further comprises a fruit juice concentrate, cane sugar, a stevia extract, polydextrose, a vegetable juice, salt, a natural flavor, a sweetener, preservative, or any combination thereof.

In a twenty first aspect, the beverage of the first aspect has from 5 calories to 20 calories per 8 ounces of beverage.

In a twenty second aspect, the beverage of the first aspect comprises:

(a) water;

(b) cannabidiol in the amount of 10 mL to 50 mL per 8 ounces of beverage;

(c) a probiotic comprising Bacillus coagulans in the amount of one billion CFU to five billion CFU per 8 ounces of beverage; and

optionally a tea extract in the amount of 0.1% to 1% by volume of the beverage.

In a twenty third aspect, the beverage of the twenty second aspect further comprises a prebiotic comprising an inulin or maltodextrin.

In a twenty fourth aspect, the beverage of twenty third aspect further comprises (1) an organic acid selected from the group of citric acid, malic acid, or a combination thereof (2) a vitamin selected from the group consisting of vitamin A, B₁, B₂, B₃, B₅, B₆, B₇, B₉, B₁₂, C, D3, E, or any combination thereof and (3) a mineral comprising a pharmaceutically-acceptable salt of calcium, chromium, copper, iron, magnesium, manganese, molybdenum, nickel, potassium, selenium, vanadium, cobalt, zinc, or any combination thereof.

In a twenty fifth aspect, the beverage of twenty fourth aspect further comprises a fruit juice concentrate, cane sugar, a stevia extract, polydextrose, a vegetable juice, salt, a natural flavor, a sweetener, preservative, or any combination thereof.

In one aspect, the beverage is composed of yogurt. “Yogurt” as used herein is a semisolid, sour-tasting food prepared from milk that has been fermented by added bacteria. In one aspect, the milk is dairy milk. Further in this aspect, the milk is from a cow or goat. Still further in this aspect, the milk can be nonfat milk, lowfat milk, or whole milk. In one aspect, “lowfat” milk is defined as milk containing 1% or 2% fat, by weight. In another aspect, milk solids can be added to the yogurt-based beverage. In still another aspect, cream can be added to the yogurt-based beverage at a point before, during, or after fermentation.

In an alternative aspect, the milk is a non-dairy milk such as, for example, soy milk, almond milk, cashew milk, coconut milk, hemp milk, oat milk, rice milk, flax milk, hazelnut milk, sunflower milk, whole grain milk, plant milk or a combination thereof.

In one aspect, the milk is dairy milk and is sourced from a cow. Further in this aspect, the cow has not been treated with hormones like recombinant bovine somatotropin (rBST, also known as recombinant bovine growth hormone or rBGH). In another aspect, the milk is nonfat milk. Further in this aspect, cream is added to the nonfat milk and the yogurt that results from fermentation of a mixture of cream and nonfat milk is lowfat yogurt. As used herein, “lowfat yogurt” refers to yogurt having from about 1 to about 5 grams of fat per 8 ounce serving, or has about 1, about 1.5, about 2, about 2.5, about 3, about 3.5, about 4, about 4.5, or about 5 grams of fat per 8 ounce serving, where any value can be a lower and upper end-point of a range (e.g., 1 g to 3 g, 2 g to 4.5 g, etc.).

In one aspect, the milk is pasteurized before fermentation. In one aspect, the bacteria used for fermentation remain alive in the yogurt-based beverage after fermentation. Further in this aspect, the bacteria used for fermentation serve as probiotics.

As used herein, a “probiotic” is a microorganism that is believed to confer health benefits when consumed. In a further aspect, a probiotic microorganism can improve the microbial balance of the intestines of the individual who consumes it. In one aspect, the probiotic is a bacterium. In an alternative aspect, the probiotic is a fungus such as, for example, a yeast ora mold.

In a twenty sixth aspect, the beverage comprises (a) yogurt, (b) a cannabinoid, and (c) a probiotic, wherein the probiotic comprises Lactobacillus bifidus and Lactobacillus acidophilus.

In a twenty seventh aspect, the yogurt of the twenty sixth aspect comprises lowfat yogurt.

In a twenty eighth aspect, the cannabinoid of the twenty sixth aspect comprises tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, cannabicitran, or any combination thereof.

In a twenty ninth aspect, the cannabinoid of the twenty sixth aspect is cannabidiol.

In a thirtieth aspect, the cannabinoid of the twenty sixth aspect is from 10 mL to 50 mL per 8 ounces of beverage.

In a thirty first aspect, the cannabinoid of the thirtieth aspect is cannabidiol.

In a thirty second aspect, the Lactobacillus bifidus of the twenty sixth aspect is from 250,000 CFU to 1,000,000 CFU per 8 ounces of beverage.

In a thirty third aspect, the Lactobacillus acidophilus of the twenty sixth aspect is from 250,000 CFU to 1,000,000 CFU per 8 ounces of beverage.

In a thirty fourth aspect, the beverage of the twenty sixth aspect further comprises a prebiotic.

In a thirty fifth aspect, the prebiotic of thirty fourth aspect comprises an inulin.

In a thirty sixth aspect, the beverage of the twenty sixth aspect further comprises an additional probiotic.

In a thirty seventh aspect, the beverage of the twenty sixth aspect further comprises one or more vitamins.

In a thirty eighth aspect, the beverage of the twenty sixth aspect further comprises one or more minerals.

In a thirty ninth aspect, the beverage of the twenty sixth aspect further comprises an organic acid or a pharmaceutically-acceptable salt thereof.

In a fortieth aspect, the beverage of the twenty sixth aspect further comprises fruit juice concentrate, cane sugar, chicory root fiber, fruit pectin, locust bean gum, milk, water, polydextrose, milk protein concentrate, tapioca starch, corn starch, calcium caseinate, potassium carbonate, vegetable juice, salt, carrageenan, whey protein concentrate, natural flavors, sweeteners, preservatives, or any combination thereof.

In a forty first aspect, the beverage of the twenty sixth aspect comprises:

(a) lowfat yogurt;

(b) cannabidiol in the amount of 10 mL to 50 mL per 8 ounces of beverage;

(c) Lactobacillus bifidus in the amount of 250,000 CFU to 1,000,000 CFU per 8 ounces of beverage; and

(d) Lactobacillus acidophilus in the amount of 250,000 CFU to 1,000,000 CFU per 8 ounces of beverage.

### Benefits of Consuming the Beverages

In one aspect, the beverages described herein are useful for fighting or reducing inflammation. “Inflammation” as used herein is part of the body's response to harmful stimuli. In the instance of injury or tissue damage, inflammation is a protective response localized to a specific area (such as, for example, redness and swelling around a burn). In another aspect, inflammation can be chronic inflammation or systemic inflammation. Further in this aspect, chronic or systemic inflammation can contribute to an overall decline in the health of the body and may be associated with diseases or conditions ranging from asthma and chronic ulcers, rheumatoid arthritis and other autoimmune diseases, periodontitis, ulcerative colitis, Crohn's disease, chronic hepatitis, chronic sinusitis, some cancers, atherosclerosis, and more. In a further aspect, consuming the beverages described herein can be useful in fighting inflammation. In one aspect, the cannabidiol contained in the beverages disclosed herein is useful in fighting inflammation. In another aspect, the probiotics and prebiotics contained in the beverages disclosed herein are also useful in fighting inflammation. In one aspect, provided herein is a method for reducing or preventing inflammation in a subject, wherein the method includes the step of consuming the any beverages disclosed herein (e.g., aspects 1 to 41 described above).

In another aspect, consumption of the beverages can correct or improve cases of dysbiosis. As used herein, “dysbiosis” refers to an imbalance of the microbial population on or inside the body, such as the intestinal tract. In some aspects, when a subject is in a state of dysbiosis, pathogenic bacteria normally present in low amounts flourish. This can lead to improper digestion, malabsorption of food and/or specific nutrients, and other illnesses and conditions due to competition between pathogenic bacteria and useful bacteria such as, for example, those that produce certain vitamins (e.g., vitamin K) from dietary precursors. In another aspect, accumulation of waste products from pathogenic bacteria is another harmful effect of dysbiosis. Prolonged dysbiosis can result from inappropriate diet, antibiotic exposure, alcohol abuse, and the like. Dysbiosis in the intestinal tract is best known, but a state of dysbiosis can be found among skin flora (leading to, for example, staph infections such as boils, impetigo, cellulitis, and staphylococcal scalded skin syndrome) and in the vagina (leading to, for examples, vaginal yeast infections). In one aspect, provided herein is a method for treating or preventing dysbiosis in a subject, wherein the method includes the step of consuming the any beverages disclosed herein (e.g., aspects 1 to 41 described above).

In another aspect, provided herein is a method for maintaining or improving digestive health, cognitive health, or immune health in a subject, wherein the method includes the step of consuming any of the beverages disclosed herein (e.g., aspects 1 to 41 described above).

Throughout this application, various publications are referenced. The disclosures of these publications in their entireties are hereby incorporated by reference into this application in order to more fully describe the compounds, compositions, and methods described herein.

Various modifications and variations can be made to the compounds, compositions, and methods described herein. Other aspects of the compounds, compositions, and methods described herein will be apparent from consideration of the specification and practice of the compounds, compositions, and methods disclosed herein. It is intended that the specification and examples be considered as exemplary.

What is claimed:

1. A beverage comprising (a) water, (b) a cannabinoid, (c) a probiotic, and (d) optionally a tea extract.

2. The beverage of claim 1, wherein the beverage includes the tea extract.

3. The beverage of claim 2, wherein the tea extract is from 0.1% to 1% by volume of the beverage per 8 ounces of beverage.

4. The beverage of claim 1, wherein the cannabinoid comprises tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, cannabicitran, or any combination thereof.

5. The beverage of claim 1, wherein the cannabinoid is cannabidiol.

6. The beverage of claim 5, wherein the cannabinoid is from 10 mL to 50 mL per 8 ounces of beverage.

7. The beverage of claim 6, wherein the cannabinoid is cannabidiol.

8. The beverage of claim 1, wherein the probiotic comprises Bacillus coagulans, Lactobacillus plantarum 299v (Lp299v), or a combination thereof.

9. The beverage of claim 1, wherein the probiotic is from one billion CFU to five billion CFU per 8 ounces of beverage.

10. The beverage of claim 1, wherein the beverage further comprises a prebiotic.

11. The beverage of claim 10, wherein the prebiotic comprises an inulin or maltodextrin.

12. The beverage of claim 1, wherein the beverage further comprises an organic acid or a pharmaceutically-acceptable salt thereof.

13. The beverage of claim 12, wherein the organic acid is from 0.01% to 0.1% by volume of the beverage per 8 ounces of beverage.

14. The beverage of claim 12, wherein the organic acid comprises malic acid or a pharmaceutically-acceptable salt thereof, citric acid or a pharmaceutically-acceptable salt thereof, or a combination thereof.

15. The beverage of claim 1, wherein the beverage further comprises one or more vitamins.

16. The beverage of claim 15, wherein the vitamin comprises vitamin A, B₁, B₂, B₃, B₅, B₆, B₇, B₉, B₁₂, C, D₃, E, or any combination thereof

17. The beverage of claim 1, wherein the beverage further comprises one or more minerals.

18. The beverage of claim 17, wherein the mineral comprises a pharmaceutically-acceptable salt of calcium, chromium, copper, iron, magnesium, manganese, molybdenum, nickel, potassium, selenium, sodium, vanadium, cobalt, zinc, or any combination thereof.

19. The beverage of claim 17, wherein the mineral comprises chromium picolinate.

20. The beverage of claim 1, wherein the beverage further comprises a fruit juice concentrate, cane sugar, a stevia extract, polydextrose, a vegetable juice, salt, a natural flavor, a sweetener, preservative, or any combination thereof.

21. The beverage of claim 1, wherein the beverage has from 5 calories to 20 calories per 8 ounces of beverage.

22. The beverage of claim 1, wherein the beverage comprises:
(d) water;
(e) cannabidiol in the amount of 10 mL to 50 mL per 8 ounces of beverage;
(f) a probiotic comprising Bacillus coagulans in the amount of one billion CFU to five billion CFU per 8 ounces of beverage; and
(g) optionally a tea extract in the amount of 0.1% to 1% by volume of the beverage.

23. The beverage of claim 22, wherein the beverage further comprises a prebiotic comprising an inulin or maltodextrin.

24. The beverage of claim 23, wherein the beverage further comprises (1) an organic acid selected from the group of citric acid, malic acid, or a combination thereof; (2) a vitamin selected from the group consisting of vitamin A, B₁, B₂, B₃, B₅, B₆, B₇, B₉, B₁₂, C, D3, E, or any combination thereof; and (3) a mineral comprising a pharmaceutically-acceptable salt of calcium, chromium, copper, iron, magnesium, manganese, molybdenum, nickel, potassium, selenium, vanadium, cobalt, zinc, or any combination thereof.

25. The beverage of claim 24, wherein the beverage further comprises a fruit juice concentrate, cane sugar, a stevia extract, polydextrose, a vegetable juice, salt, a natural flavor, a sweetener, preservative, or any combination thereof.

26. A method for treating or preventing inflammation in a subject comprising consuming the beverage of claim 1.

27. A method for treating or preventing dysbiosis in a subject comprising consuming the beverage of claim 1.

28. A beverage comprising (a) yogurt, (b) a cannabinoid, and (c) a probiotic, wherein the probiotic comprises Lactobacillus bifidus and Lactobacillus acidophilus.

29. The beverage of claim 28, wherein the yogurt comprises lowfat yogurt.

30. The beverage of claim 28, wherein the cannabinoid comprises tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, cannabicitran, or any combination thereof

31. The beverage of claim 28, wherein the cannabinoid is cannabidiol.

32. The beverage of claim 28, wherein the cannabinoid is from 10 mL to 50 mL per 8 ounces of beverage.

33. The beverage of claim 32, wherein the cannabinoid is cannabidiol.

34. The beverage of claim 28, wherein the Lactobacillus bifidus is from 250,000 CFU to 1,000,000 CFU per 8 ounces of beverage.

35. The beverage of claim 28, wherein the Lactobacillus acidophilus is from 250,000 CFU to 1,000,000 CFU per 8 ounces of beverage.

36. The beverage of claim 28, wherein the beverage further comprises a prebiotic.

37. The beverage of claim 36, wherein the prebiotic comprises an inulin.

38. The beverage of claim 28, wherein the beverage further comprises an additional probiotic.

39. The beverage of claim 28, wherein the beverage further comprises one or more vitamins.

40. The beverage of claim 28, wherein the beverage further comprises one or more minerals.

41. The beverage of claim 28, wherein the beverage further comprises an organic acid or a pharmaceutically-acceptable salt thereof.

42. The beverage of claim 28, wherein the beverage further comprises fruit juice concentrate, cane sugar, chicory root fiber, fruit pectin, locust bean gum, milk, water, polydextrose, milk protein concentrate, tapioca starch, corn starch, calcium caseinate, potassium carbonate, vegetable juice, salt, carrageenan, whey protein concentrate, natural flavors, sweeteners, preservatives, or any combination thereof.

43. The beverage of claim 28, wherein the beverage comprises:
(e) lowfat yogurt;
(f) cannabidiol in the amount of 10 mL to 50 mL per 8 ounces of beverage;
(g) Lactobacillus bifidus in the amount of 250,000 CFU to 1,000,000 CFU per 8 ounces of beverage; and
(h) Lactobacillus acidophilus in the amount of 250,000 CFU to 1,000,000 CFU per 8 ounces of beverage.

44. A method for treating or preventing inflammation in a subject comprising consuming the beverage of claim 28.

45. A method for treating or preventing dysbiosis in a subject comprising consuming the beverage of claim 28.
